(19) United States
(12) Patent Application Publication
(10) Pub. No.: US 2017/0143525 A1
(43) Pub. Date: May 25, 2017

(54) Article With Support Strips

(71) Applicant: Nicholas Matfus, New York, NY (US)

(72) Inventor: Nicholas Matfus, New York, NY (US)

(21) Appl. No.: 14/999,666

(22) Filed: Jun. 10, 2016

Related U.S. Application Data
(60) Provisional application No. 62/386,282, filed on Nov. 24, 2015.

(51) Int. Cl. A61F 5/01; A41D 13/06; A41D 13/08

(52) U.S. Cl. CPC A61F 5/0109; A61F 5/0118; A61F 5/0111; A41D 13/08; A41D 13/06; A41D 2400/32; A41D 2500/50; A41D 2500/20

(57) ABSTRACT

The present invention provides for an article and more specifically, to a garment with support strips such as kinesiology tape and related methods of manufacturing.

### RELATED APPLICATION

This application is related to U.S. Patent Provisional Application Ser. No. 62/386,282 filed on Nov. 24, 2015 entitled “Article with Support Strips”.

### BACKGROUND OF THE INVENTION

### Field of the Invention

This invention relates to an article and more specifically, to a garment with support strips such as kinesiology tape.

### SUMMARY OF THE INVENTION

In one embodiment, the present invention provides for an article comprising: a fabric forming a garment designed to cover a portion of a user's body, and the garment further comprises at least one support strip. In another embodiment, the article comprises a sleeve. In yet another embodiment, the support strip comprises kinesiology tape. In still another embodiment, the support strip is incorporated into said fabric.

In still yet another embodiment, the article further comprises a plurality of support strips. In a further embodiment, the garment is constructed of a material that is different from than the support strip's material. In another further embodiment, the garment is constructed of a material selected from a group consisting essentially of cotton, polyester, synthetics, wool, nylons, natural materials, rayon, textile fibers, blends and combinations thereof.

In yet a further embodiment, the support strip is constructed of a material selected from a group consisting essentially of cotton, synthetics, plastics, elastomers, natural materials, textile fabrics, rubber, blends and combinations thereof.

In still a further embodiment, the strips are position to support the user's joints. In still yet a further embodiment, the plurality of the support strips are positioned to intersect one another. In another embodiment, the plurality of support strips are positioned to be parallel to one another. In yet another embodiment, the plurality of support strips are positioned to be symmetrical. In still another embodiment, the plurality of support strips are positioned to be asymmetrical. In still yet another embodiment, the plurality of support strips are of uniform length and width. In a further embodiment, the plurality of support strips are of varying lengths and widths. In another further embodiment, the plurality of supports strips have uniform length and varying width. In yet another further embodiment, the plurality of supports strips have uniform width and varying length.

In still another further embodiment, the position of the support strips on the garment is adjustable. In still yet another further embodiment, the garment is constructed of compression materials. In another embodiment, the support strip is printed onto the fabric. In yet another embodiment, the support strip is sewn into the garment. In still another embodiment, the support strip is inserted into the garment.

In still yet another embodiment, a portion of said garment is curved to support the user's joint. In another embodiment, the garment further comprises materials that produce beneficial biologic properties that include ion producing materials and far infrared (FIR) materials.

In another further embodiment, the materials that produce beneficial properties are incorporated into said fabric by methods consisting essentially of weaving, infusion, coating, sewing, dipping, and spraying and combinations thereof.

In yet a further embodiment, the materials that produce beneficial properties are selected from a group consisting essentially of metals, ceramics, ion producing substances, FIR producing substances, and mixtures and combinations thereof.

In still a further embodiment, the open edges of the garment are reinforced with elastic materials.

In still yet a further embodiment, the present invention relates to an article manufactured by a method comprising: attaching at least one support strip onto a fabric which is formed into a garment.

In another embodiment, the support strip is attached to said fabric by methods consisting essentially of weaving, infusion, coating, sewing, dipping, and imprinting and combinations thereof. In yet another embodiment, the method of manufacturing the article further comprises incorporating materials that produce beneficial properties onto the garment and/or incorporating materials that produce beneficial properties onto the support strip.

In a further embodiment, the present invention relates to a method of manufacturing a garment, said method comprising: attaching at least one support strip onto a fabric which is formed into a garment.

In another further embodiment, the support strip is attached to said fabric by methods consisting essentially of weaving, infusion, coating, sewing, dipping, and imprinting and combinations thereof.

In yet a further embodiment, the method further comprises incorporating materials that produce beneficial biologic properties into said garment.

In still a further embodiment, the method further comprises attaching said plurality of support strips onto said garment. In still yet a further embodiment, the garment is constructed of compression materials and the support strip comprises kinesiology tape.

In another embodiment, the article can be a garment, a sleeve, a brace, or a support.

### BRIEF DESCRIPTION OF THE DRAWINGS

The accompanying drawings are included to provide a further understanding of the present invention. These drawings are incorporated in and constitute a part of this specification, illustrate one or more embodiments of the present invention and together with the description, serve to explain the principles of the present invention.

FIG. 1 is a perspective view of one of the embodiments of the present invention wherein the article is an elbow sleeve;

FIG. 2 is a perspective view of FIG. 1 showing the elbow sleeve in use;

FIG. 3 is a perspective view of another embodiment wherein the article is a knee brace;

FIG. 4 is a perspective view of FIG. 3 showing the brace in use;

FIG. 5 is a perspective view of a further embodiment wherein the article is an ankle support; and

FIG. 6 is a perspective view of FIG. 5 showing the ankle support in use.

Among those benefits and improvements that have been disclosed, other objects and advantages of this invention will become apparent from the following description taken in conjunction with the accompanying drawings. The drawings constitute a part of this specification and include exemplary embodiments of the present invention and illustrate various objects and features thereof.

### DETAILED DESCRIPTION OF THE INVENTION

As required, detailed embodiments of the present invention are disclosed herein; however, it is to be understood that the disclosed embodiments are merely exemplary of the invention that may be embodied in various forms. The figures are not necessarily to scale, some features may be exaggerated to show details of particular components. Therefore, specific structural and functional details disclosed herein are not to be interpreted as limiting, but merely as a basis for the claims and as a representative basis for teaching one skilled in the art to variously employ the present invention.

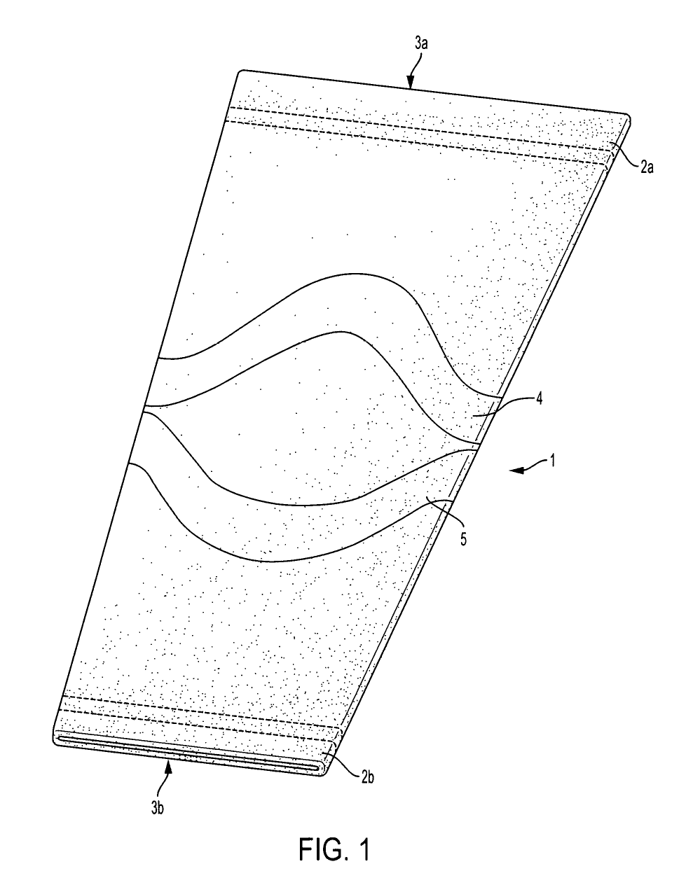

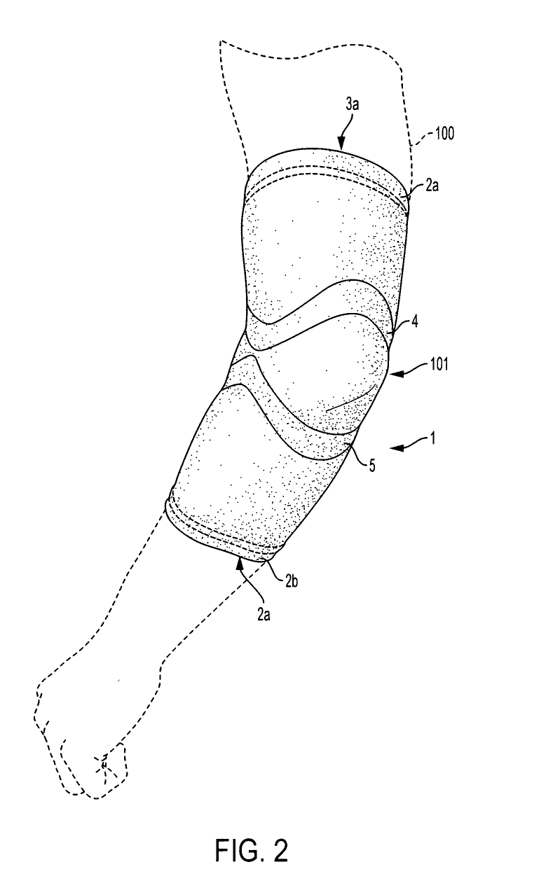

FIGS. 1-2 illustrate the article used as a sleeve 1. The sleeve 1 has opposing ends, 2a and 2b respectively and two openings, 3a and 3b respectively. The openings, 3a and 3b, of the sleeve 1 is designed to slide into a user's arm 100 and fit around the elbow area. There are at least two support strips, 4 and 5. The supports strips, 4 and 5, can be kinesiology tape and are strategically placed to support a user's elbow 101.

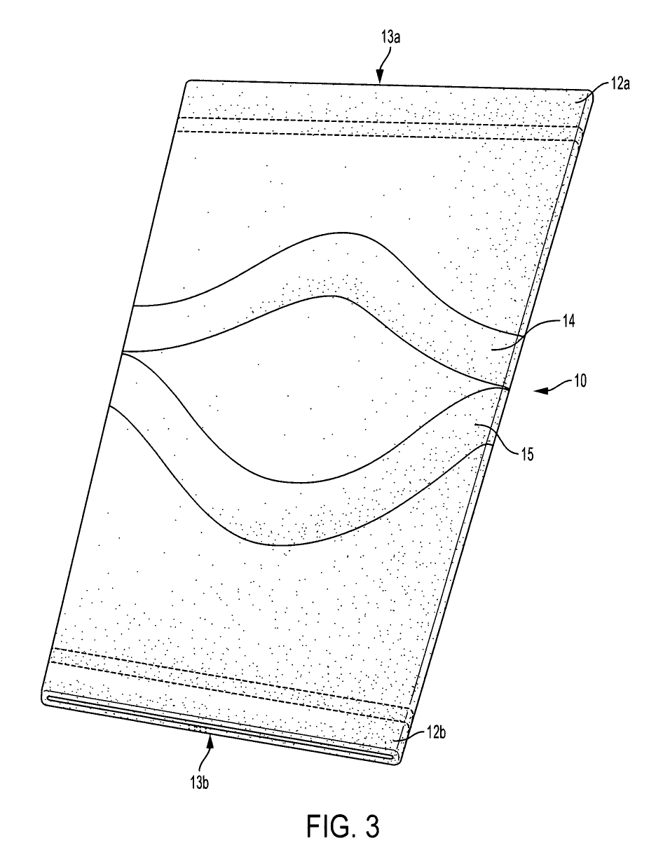

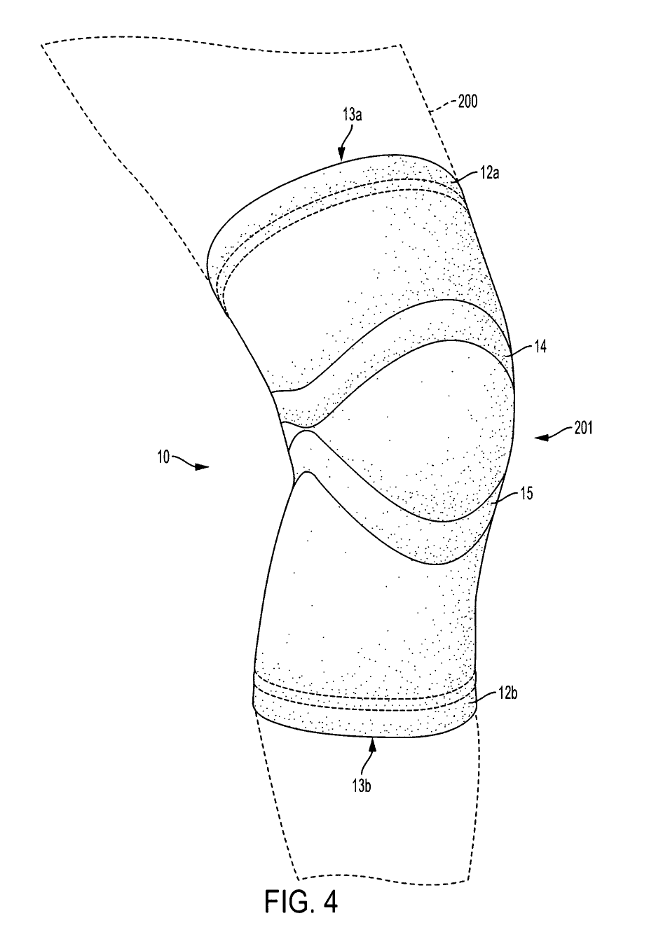

FIGS. 3-4 illustrate the article used as a brace 10. The sleeve 10 has opposing ends, 12a and 12b respectively and two openings, 13a and 13b respectively. The openings, 13a and 13b, of the brace 10 is designed to slide over a user's leg 200 and fit around the knee area. There are at least two support strips, 14 and 15. The supports strips, 14 and 15, can be kinesiology tape and are strategically placed to support a user's knee 201.

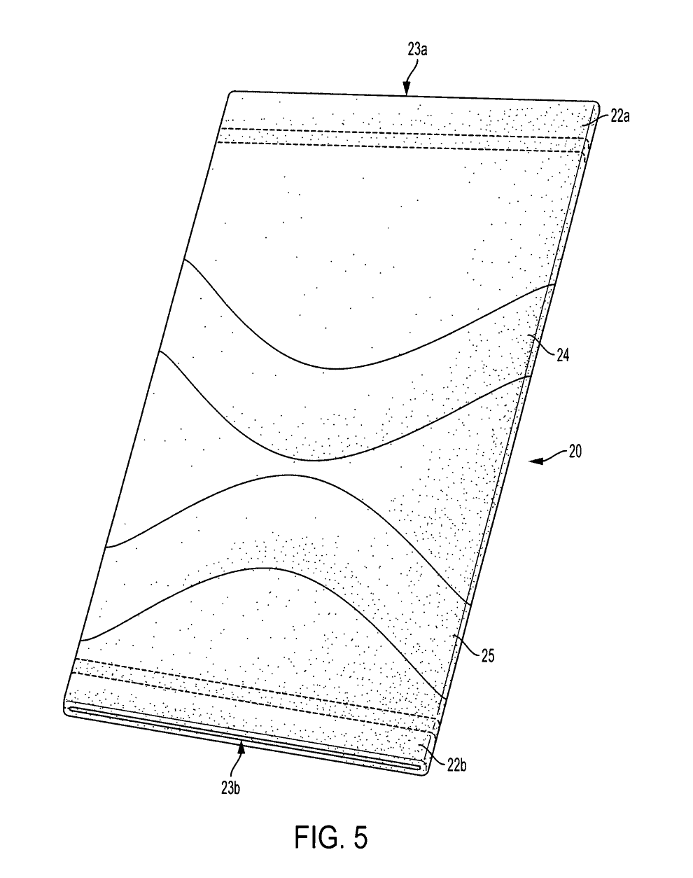

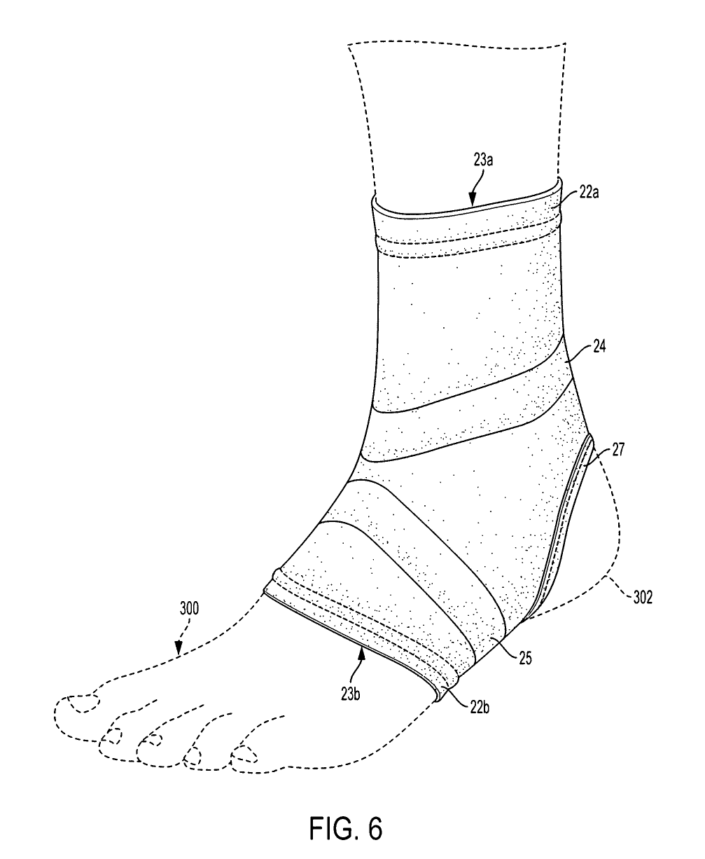

FIGS. 5-6 illustrate the article used as an ankle support 20. The ankle support 20 has opposing ends, 22a and 22b respectively and two openings, 23a and 23b respectively. In between the openings, 23a and 23 b, there is a cut-out 27. The openings, 23a and 23b, of the ankle support 20 is designed to slide over a user's foot 300 and fit around the foot area. There are at least two support strips, 24 and 25. The supports strips, 24 and 25, can be kinesiology tape and are strategically placed to support a user's foot 300. The cut-out 27 is designed to be situated at the user's heel 302.

The subject matter of this application is marketed as Copper Fit Pro™, which utilizes a technology that combines Thermoplastic Polyurethane (TPU) with hot melt adhesive films. The combination allows the sports sleeves and garments to incorporate enhanced support around an individual user's joints. The TPU being bonded to the sleeve allows the sleeve to give two different levels of support to the user. A first level is that of a more traditional compression sleeve, this support surrounds the entire joint and provides support for the adjacent muscles. The second level is akin to more traditional methods of taping a joint that is either injured or is exposed to excess stress during a particular athletic event. Therefore, the second level of support provides targeted support specifically for the joint the sleeve covers on the user. Thus, the sleeve in the instance application allows a user to slip on or off a compression sleeve that incorporates TPU tape. This allows for enhanced support. It also allows for a quickly applied and reusable support for repeated uses.

The TPU has a thickness from a range of about 0.05 to 0.50 mm. The TPU has a softening range between about 180 to 220° F. The melting temperature of the TPU is between 260-300° F.

The TPU is made to the specifications of the sleeve depending on which part of the body the sleeve is designed to support. In particular, it is pre-slit with a laser cutting machine to the specifications required for the unique designs of the knee, elbow, wrist, and ankle sleeves. The sleeves also utilize fabric lamination welding method. This welding method allows for precision sleeves to be produced. The welding requires ultrasonic welding equipment, industrial hot air blowers, industrial strength irons, and a heat press machine to create and manufacture the sleeves. The welding also utilizes bonding material such as polyester.

Numerous modifications and variations of the present invention are possible in light of the above teachings. It is therefore to be understood that within the scope of the attendant claims attached hereto, this invention may be practiced otherwise than as specifically disclosed herein.

What is claimed is:

1. An article comprising: a fabric forming a garment designed to cover a portion of a user's body, said garment further comprising at least one support strip.

2. The article of claim 1 comprises a sleeve.

3. The article of claim 1 wherein said support strip comprises kinesiology tape.

4. The article of claim 1 wherein said support strip is incorporated into said fabric.

5. The article of claim 1 further comprises a plurality of support strips.

6. The article of claim 1 wherein said garment is constructed of a material that is different from than the support strip's material.

7. The article of claim 6 wherein said garment is constructed of a material selected from a group consisting essentially of cotton, polyester, synthetics, wool, nylons, natural materials, rayon, textile fibers, blends and combinations thereof.

8. The article of claim 6 wherein said support strip is constructed of a material selected from a group consisting essentially of cotton, synthetics, plastics, elastomers, natural materials, textile fabrics, rubber, blends and combinations thereof.

9. The article of claim 5 wherein said strips are position to support the user's joints.

10. The article of claim 5 wherein said plurality of said support strips are positioned to intersect one another.

11. The article of claim 5 wherein said plurality of said support strips are positioned to be parallel to one another.

12. The article of claim 5 wherein said plurality of said support strips are positioned to be symmetrical.

13. The article of claim 5 wherein said plurality of said support strips are positioned to be asymmetrical.

14. The article of claim 5 where said plurality of said support strips are of uniform length and width.

15. The article of claim 5 wherein said plurality of said support strips are of varying lengths and widths.

16. The article of claim 5 wherein said plurality of said supports strips have uniform length and varying width.

17. The article of claim 5 wherein said plurality of said supports strips have uniform width and varying length.

18. The article of claim 5 wherein the position of said support strips on the garment is adjustable.

19. The article of claim 5 wherein garment is constructed of compression materials.

20. The article of claim 1 wherein said support strip is printed onto said fabric.

21. The article of claim 1 wherein said support strip is sewn into said garment.

22. The article of claim 1 wherein said support strip is inserted into said garment.

23. The article of claim 1 wherein a portion of said garment is curved to support the user's joint.

24. The article of claim 1 wherein said garment further comprises materials that produce beneficial biologic properties.

25. The article of claim 24 wherein said materials that produce beneficial properties include ion producing materials.

26. The article of claim 24 wherein said materials that produce beneficial properties include far infrared (FIR) materials.

27. The article of claim 24 wherein said materials that produce beneficial properties are incorporated into said fabric by methods consisting essentially of weaving, infusion, coating, sewing, dipping, and spraying.

28. The article of claim 24 wherein said materials that produce beneficial properties are selected from a group consisting essentially of metals, ceramics, ion producing substances, FIR producing substances, and mixtures and combinations thereof.

29. The article of claim 1 wherein the open edges of said garment are reinforced with elastic materials.

30. An article manufactured by a method comprising: attaching at least one support strip onto a fabric which is formed into a garment.

31. The article of claim 30 wherein said support strip is attached to said fabric by methods consisting essentially of weaving, infusion, coating, sewing, dipping, and imprinting and combinations thereof.

32. The article of claim 30 further comprising incorporating materials that produce beneficial properties onto said garment.

33. The article of claim 30 further comprising incorporating materials that produce beneficial properties onto said support strip.

34. A method of manufacturing a garment, said method comprising: attaching at least one support strip onto a fabric which is formed into a garment.

35. The method of claim 34 wherein said support strip is attached to said fabric by methods consisting essentially of weaving, infusion, coating, sewing, dipping, and imprinting and combinations thereof.

36. The method of claim 34 further comprising incorporating materials that produce beneficial biologic properties into said garment.

37. The method of claim 36 wherein said materials that produce beneficial properties are selected from a group consisting essentially of metals, ceramics, ion producing substances, FIR producing substances, and mixtures and combinations thereof.

38. The method of claim 34 further comprising providing a plurality of support strips.

39. The method of claim 38 further comprising attaching said plurality of support strips onto said garment.

40. The method of claim 34 wherein said garment is constructed of compression materials and said support strip comprises kinesiology tape.
